(19) United States
(12) Patent Application Publication
(10) Pub. No.: US 2007/0299323 A1
(43) Pub. Date: Dec. 27, 2007

(54) Apparatus for measuring one or more physiological functions of a body and a method using the same

(76) Inventors: Martijn Wilco Arns, Nijmegen (NL); Gerardus Alexander Slootweg, Elst (NL); Dirk Henry Kleinnijenhuis, Nijmegen (NL)

Correspondence Address: MERCHANT & GOULD PC, P.O. BOX 2903, MINNEAPOLIS, MN, 55402-0903, US

(21) Appl. No.: 11/476,227

(22) Filed: Jun. 27, 2006

(51) Int. Cl. A61B 5/00

(52) U.S. Cl. 600/301

(57) ABSTRACT

The present invention relates to an apparatus for measurement of a physiological function of a body comprising; physiological function detection means responsive to a physiological signal and capable of generating an analog output signal corresponding to the physiological signal; an analog-digital converter capable of receiving the unamplified analog output signal and capable of generating a digital output signal corresponding to the unamplified analog output signal and control means capable of receiving and communicating the digital output signal.

### BACKGROUND OF THE INVENTION

The present invention generally relates to an apparatus for measuring one or more physiological functions of a body, such as a human body, and a method using said apparatus for measurement of one or more physiological functions. In some preferred embodiments, the apparatus according to the present invention is used to measure the heart rate or the emotional state of a human body or a combination thereof. In a specially preferred embodiment, the present invention relates to a watch-like apparatus for measuring, on the wrist, the heart rate and emotional state of a human individual indicative for the cardiac status or emotional status of said individual.

Measurement of physiological functions per se is known in the art. Examples of such measurements are measuring muscle activity such as the heart rate, blood pressure, brain activity, organ or tissue status or condition, etc.

In general, such physiological functions are measured or determined using a sensor device responsive to a physiological signal relating to the physiological function such as electric fields generated by muscle activity such as the heart rate, blood pressure indicative of the heart rate and/or activity, blood glucose levels indicative for diabetes, electric fields generated by brain activity indicative for brain functioning, the presence/absence and/or concentration of organic and/or inorganic molecules and compounds such as metabolites, hormones, proteins, lipids or salts indicative for the status or condition of an organ or tissue, etc.

The response of the sensor device is usually an analog electric output signal, corresponding to the physiological signal, i.e., the analog electric output signal comprises information, which information could be the presence or absence of the signal or the strength of the signal, indicative for the physiological function. Such sensor devices are well known to the skilled person and are readily available in the art.

The analog electric output signal, generated by the sensor device, is in most cases too low, for example in the order of millivolts, for direct further processing. Such processing could comprises converting or arranging the generated electric analog output signal into for a physician or patient readable or usable information.

Therefore, in most cases, the apparatuses for measuring physiological functions according to the prior art comprise, besides the above sensor device, one or more amplifiers, increasing the strength of the electric analog output signal before it is further processed into, for example, physician or patient readable or usable information.

However, the use of such one or more amplifiers usually results in adding additional background noise to the analog signal decreasing the quality of the outputted analog signal. This could result in a loss of valuable physiological information comprised in the original signal.

Further, the use of additional amplifier components and their corresponding supporting structures and components in the apparatuses according to the prior art results in an increase of the necessary dimensions of such apparatuses while in most cases it is desired that these apparatuses are as small as possible.

Furthermore, additional amplifier components, and their corresponding supporting structures and components, add to the total costs of these apparatuses for measuring physiological functions.

Because of the above reasons, one of the goals of the present invention to provide an apparatus for measurement of a physiological function of a body wherein the use of said amplifier components, and their corresponding supporting structures and components, are avoided or even eliminated.

Other goals and objectives of the present invention will be apparent to the skilled person after reading the specification of the present invention as provided herein and/or the appended claims and/or by practicing the present invention.

### SUMMARY OF THE INVENTION

The present invention relates to an apparatus for measurement of a physiological function of a body comprising:
(i) physiological function detection means responsive to a physiological signal and capable of generating an analog output signal corresponding to the physiological signal;
(ii) an analog-digital (AD) converter capable of receiving the unamplified analog output signal and capable of generating a digital output signal corresponding to the unamplified analog output signal;
(iii) control means capable of receiving and communicating the digital output signal.

According to a preferred embodiment, the present invention relates to the above specified apparatus further comprising (iv) movement detection means capable of generating a movement signal corresponding with movement of the body or a part thereof and said controls means are further capable of receiving the movement signal.

In a more preferred embodiment, the analog-digital (AD) converter according to the present invention is a high resolution analog-digital (AD) converter of 16 bit or higher, more preferably a high resolution analog-digital (AD) converter of 24 bit or higher.

According to the present invention, it is preferred that the unamplified analog output signal is directly communicated to the analog-digital (AD) converter or the unamplified analog output signal is indirectly communicated through a low pass pre-sampling filter to the analog-digital (AD) converter.

According to one preferred aspect of the present invention, the physiological function detection means are suitable for detecting pulse oximetry and/or (photo)plethysmography and comprise an infrared or red light emitting source and a photodiode capable of detecting infrared or red light.

According to a preferred embodiment of the above aspect of the present invention, the apparatus for measurement of a physiological function of a body comprises two or more, preferably five or more, and most preferably seven photodiodes capable of detecting infrared or red light.

According to another preferred aspect of the present invention, the physiological function detection means are suitable for detecting the galvanic skin response (GSR) and comprise two or more electrodes capable of measuring the resistance of the skin between the two or more electrodes.

According to yet another preferred aspect of the present invention, the apparatus for measurement of a physiological function comprises two or more physiological function detection means each responsive to a different physiological signal, two or more analog-digital (AD) converters, or a multichannel AD converter, corresponding with the two or more physiological signal detection means, and receiving means capable of receiving and communicating the two or more digital output signals.

A particularly preferred embodiment of the above aspect of the present invention relates to an apparatus for measurement of a physiological function of a body comprising first physiological function detection means suitable for detecting pulse oximetry and/or (photo)plethysmography comprising an infrared or red light emitting source and one or more photodiodes capable of detecting infrared or red light; and second physiological function detection means suitable for detecting the galvanic skin response (GSR) comprising two or more electrodes capable of measuring the resistance of the skin between the two or more electrodes.

The above described apparatuses, aspects of the present invention and preferred embodiments thereof provide several advantages over the prior art devices such as, besides other advantages, avoiding or even eliminating the use of signal information decreasing, expensive and space consuming amplifier components and their corresponding supporting structures and components.

Therefore, the apparatuses according to the present invention are advantageously used in a method for measuring, preferably measuring comprises multiple or continuous measurements of the one or more physiological functions, one or more physiological functions of a body comprising measuring of the one or more physiological functions using an apparatus according to the present invention.

The apparatuses according to the present invention are also, for the same reasons, advantageously used in a method for analysis of one or more physiological functions of a body comprising multiple or continuous measuring of the one or more physiological functions using an apparatus according to the present invention, communicating the measurements to an external source and analyzing the measurements using the external source.

### BRIEF DESCRIPTION OF THE FIGURES

The present invention will be apparent to the skilled person by reading the following disclosure, with reference to the attached figure, wherein:

FIG. 1 is a schematic diagram of a preferred embodiment of the present invention capable of measuring pulse oximetry and/or (photo)plethysmography and the galvanic skin response (GSR).

FIG. 2 is a possible selection algorithm for selecting the most suitable signals when multiple analog output signals are generated.

### DETAILED DESCRIPTION OF THE INVENTION

The present invention relates to an apparatus for measurement of a physiological function of a body comprising:
(iv) physiological function detection means responsive to a physiological signal and capable of generating an analog output signal corresponding to the physiological signal;
(v) an analog-digital (AD) converter capable of receiving the unamplified analog output signal and capable of generating a digital output signal corresponding to the unamplified analog output signal;
(vi) control means capable of receiving and communicating the digital output signal.

The term “physiological function” as used herein refers to any physiological function of the body, such as a human body, including for example, but not limited thereto, muscle activity, brain activity, eye activity, blood saturation, blood pressure, heart rate via plethysmography, Galvanic Skin Response (GSR, or Electrodermal activity, skin conductance level), organ or tissue status or condition, etc.

The apparatus according to the present invention comprises physiological function detection means responsive to a physiological signal and capable of generating an analog output signal corresponding to the physiological signal.

Non-limiting examples of physiological signals are electric fields generated by muscle activity such as the heart rate, blood pressure indicative of the heart rate and/or activity, blood glucose levels indicative for diabetes, electric fields generated by brain activity, the presence and/or concentration of organic and inorganic molecules such as metabolites, hormones, proteins, lipids or salts indicative for the status or condition of an organ or tissue, etc.

The physiological detection means are readily and commercially available in the prior art. Examples of such means are EEG measuring devices; EEG amplifiers, ECG amplifiers, EMG amplifiers, plethysmographs, heart rate monitors etc.

The analog output signal according to the present invention is an analog electric signal, usually in the range of millivolts, generated by the physiological detection means in response to a physiological signal.

The generated analog output signal is said to be corresponding with the physiological signal if information is contained therein is indicative for the presence and/or value of the physiological signal. Such information could be the presence or absence of the analog output signal and/or the value of the analog output signal and/or the pattern or phase of the analog output signal.

According to the present invention, in contrast with a device according to the prior art, the analog output signal is communicated, without passing any amplifier, thus unamplified, to an analog-digital (AD) converter capable of receiving the unamplified analog output signal and capable of generating a digital output signal corresponding to the unamplified analog output signal

Suitable analog-digital (AD) converters according to the present invention can be easily identified by the skilled person using the information which is set out herein.

For example, a suitable analog-digital (AD) converter according to the present invention should be able to convert the analog output signal into a digital signal whereby the information corresponding to the physiological signal is at least partially, or preferably completely, derivable from the outputted digital signal using appropriate means such as a microcomputer or microcontroller.

This can for example be achieved by using an analog-digital (AD) converter which is capable of dividing the analog output signal in sufficient equal intervals, such as for example 65536 intervals, thereby preserving small millivolt changes in the analog output signal possibly indicative for at least some aspects, preferably all aspects, of the physiological signal.

According to the present invention, preferably an analog-digital (AD) converter is used which is a high resolution analog-digital (AD) converter of 16 bit or higher, thus 65536 intervals or higher.

Even more preferably, an analog-digital (AD) converter is used which is a high resolution analog-digital (AD) converter of 24 bit or higher, such as preferably a 24 bit high resolution analog-digital (AD) converter, thus 16777216 intervals or higher.

The apparatus according to the invention also comprises control means capable of receiving and communicating the digital output signal. These control means are generally known and commercially available in the art and usually sold under the designation of controller or microcontroller. Examples of such control means are a ATmega32 microcontroller produced by Atmel.

For receiving, the control means according to the present invention usually comprise an integrated receiving assembly for example in the form of a receiving port for connection to the analog-digital converter and an integrated communicating assembly in the form of an output port or transmitting device such as Bleutooth, infrared, or other remote communication.

Other components could also be present such as memory components or computing components for handling and/or processing of, for example, the digital output signal before it is communicated to, for example, an external source such as a microcomputer for further processing and handling. However, a microcomputer or processor chip can also be integrated in the control means or in the apparatus according to the present invention.

The physiological function detection means, the analog-digital (AD) converter, and the control means could be readily integrated in one circuitry board present in the apparatus according to the present invention. However, they could also be separately provided, or partially integrated, in the apparatus.

It is contemplated within the scope of the present invention that the apparatus also comprises display means for indicating, for example, the status of the apparatus and/or the different generated signals and/or processed derivatives thereof.

There is no limitation according to the present invention with respect to the type of connection which is used to allow communication or transmission of the signals between the different means according to the present invention.

Such connections could for example be partially or completely embodied in traditional wiring or partially or completely in the form of transmission of suitable wavelengths such high frequency waves, for example radio waves, or light either visible or not visible such as infrared light.

However, according to the invention, the outputted analog signal is first (i) communicated, without amplification, to the analog-digital converter and subsequently (ii) the digital output signal is communicated to the control means.

In a preferred embodiment of the present invention, the analog output signal is directly communicated to the analog-digital converter. Under the term “directly” as used herein should be understood without passing any devices or components which substantially modify, change or alter the analog output signal arriving at the analog-digital converter.

The term “substantially” as used herein means an analog output signal arriving at the analog-digital converter which retained at least 50%, preferably 70%, more preferably 80%, even more preferably 90% and most preferably 100% of his signal strength in the relevant frequency domain compared to the same analog output signal leaving the physiological function detection means.

In another preferred embodiment of the present invention, the unamplified analog output signal is indirectly communicated through a low pass pre-sampling filter to the analog-digital (AD) converter. By using a low pass pre-sampling filter, such as a first order low pass pre-sampling filter, high frequencies are reduced or eliminated from the analog output signal.

These high frequencies usually do not contain information corresponding to the generated physiological signal, hence can be safely filtered from the signal without losing information. By doing this, a digital output signal is obtained with a higher resolution than without filtering of the analog output signal. It should however be noted that also in this embodiment, the analog output signal arrives unamplified, as defined above, at he analog-digital converter.

In a particular preferred embodiment of the present invention, the apparatus further comprising movement detection means capable of generating a movement signal corresponding with movement of the body or a part thereof, such as a arm or joint, and the controls means are further capable of receiving the movement signal.

This embodiment provides the additional advantage that, amongst others, the quality of the measured physiological signal is dramatically enhanced.

Most of the physiological measurements are very susceptible to movement artefacts. Therefore, it is very difficult to measure and subsequently analyze these signals in real-life meaning without subjection the individual to an artificial environment like an hospital.

In order to reduce motion artefacts in physiological signals, several techniques are already in use. For example, complex software algorithms are already developed to reduce and/or correct for signal artefacts induced by movement. Despite of all these techniques, it is still very difficult to provide accurate reliable measurements during gross movements. However, if a measurement is obtained when no movement is detected or movement is minimal meaning below a predetermined threshold-level, movement artefact reduction techniques are not necessary, and the physiological signal can be measured and analysed accurately and reliably.

This will also reduce the likelihood of wrong interpretation of the physiological signal due to movement artefacts. In applications where no continuous data is required, but only a limited number of measurements in a certain time slot are needed, the measurements can be scheduled on basis of movement detection with for example an accelerometer, gyroscope, etc. Measurements will only be taken when movement is below a certain critical threshold, indicating that a correct reliable reading of the physiological signal can be done.

However, according to the present invention, it is not necessary that the measurement itself is only performed when the detected movement is below a predetermined threshold value. It is for example possible to couple, using the control means, movement data tot the digital output signal. In case it is determined that the corresponding movement data indicates that the measurement is unreliable, such data can be ignored during the subsequent processing of the data obtained by, for example, an external source such as a microcomputer.

Other embodiments can be envisaged such as only activating the physiological detection means the moment it is determined that the movement signal is below a certain threshold or denying the physiological output signal or the digital output signal access to the analog-digital converter or the control means, respectively.

The above are some exemplary embodiments of how the detection of the movement can be incorporated in the device according to the invention. Other embodiments can be readily envisaged by the skilled person and are considered to fall within the scope of the present invention as long as movement detection is combined with unamplified physiological output signal processing through the analog-digital converter.

In a particular preferred embodiment, the present invention relates to physiological function detection means suitable for detecting pulse oximetry and/or (photo)plethysmography and comprise an infrared or red light emitting source and a photodiode capable of detecting infrared or red light.

These physiological detection means are well known in the art and provide in combination with the apparatuses according to the present invention, an until hitherto not achieved level of measurement of pulse oximetry and/or (photo)plethysmography.

Pulse oximetry is a non-invasive method which allows health care providers to monitor the oxygenation of a patient's blood.

In general, a sensor is placed on a part of the patient's anatomy, and red and/or infrared light is passed through the body. Based upon the ratio of absorption of the red and/or infrared light caused by the difference in color between oxygen-bound (red) and unbound (blue) hemoglobin in the capillary bed, an approximation of oxygenation can be made

(Photo)plethysmography is based on the determination of the optical properties of a selected skin area. For this purpose, non-visible infrared light is emitted into the skin. More or less light is absorbed, depending on the blood volume in the skin. Consequently, the backscattered light corresponds with the variation of the blood volume. Blood volume changes can then be determined by measuring the reflected light and using the optical properties of tissue and blood.

The principle of measuring these physiological signals is that by using pulse oximetry or (photo)plethysmography, the pulsation of the blood can be measured thereby peroviding information about the heart rate indicative for the condition of the heart.

In another particular preferred embodiment, the present invention relates to an apparatus comprising two or more, preferably five or more, and most preferably seven photodiodes capable of detecting infrared or red light.

With respect to, for example, pulse oximetry and/or (photo)plethysmography measurements, by using multiple photodiodes, a further cleaning of the data can be obtained and ease of use guaranteed (e.g. due to displacement of the apparatus according to the present invention or body, the chance of picking up at least one good signal is bigger then with 1 photodiode.) Furthermore, these multiple photodiodes, i.e., two or more, preferably five or more, and most preferably seven, enable processing techniques to further clean the data such as source localization techniques, averaging all channels, triangulation, etc.

The physiological output signals generated by these photodiodes can be simultaneously or in sequence inputted in the analog-digital (AD) converter according to the present invention. Subsequent selection or processing, such as averaging, of the digital output signals can be performed in the control means or using additional components in the apparatus itself or an external source such as a microcomputer.

In another preferred embodiment, the apparatus according to the present invention comprises physiological function detection means suitable for detecting the galvanic skin response (GSR) and comprise two or more electrodes capable of measuring the resistance of the skin between the two or more electrodes.

The galvanic skin response (GSR) is related to the emotional condition of an individual, such as anxiety, stress, fear, etc. It is well known in the art that these factors are influencing the cardiacal conditions of an individual, hence providing an physiological signal helpful for diagnosing, by for example a physician, of the physiological condition of the heart.

Because of this, it is especially preferred to combine both above physiological detection means, i.e., pulse oximetry and/or (photo)plethysmography and galvanic skin response (GSR) in one apparatus according to the present invention.

Hence, the present invention also relates to an apparatus comprising first physiological function detection means suitable for detecting pulse oximetry and/or (photo)plethysmography comprising an infrared or red light emitting source and one or more photodiodes capable of detecting infrared or red light; and second physiological function detection means suitable for detecting the galvanic skin response (GSR) comprising two or more electrodes capable of measuring the resistance of the skin between the two or more electrodes.

However, the advantage of combining the measurement of two or more, preferably physiologically related, physiological detection means in one apparatus is not restricted to the above embodiment. The skilled person will immediately recognize that the above benefits can also be provided when other types of physiological conditions are assessed such as brain activity by combining blood pressure with the electric fields measurements, diabetes by combining glucose and insulin levels, hormonal deficiencies by combining urea concentrations and the galvanic skin response (GSR), immunological status by combining white blood cell counts and the blood pressure and galvanic skin response (GSR), etc.

Therefore, the present invention also relates, in a preferred embodiment, more general to an apparatus comprising two or more physiological function detection means each responsive to a different physiological signal, two or more analog-digital (AD) converters or a multichannel analog-digital (AD) converter capable of substituting for the two or more analog-digital (AD) converters, corresponding with the two or more physiological signal detection means, and receiving means capable of receiving and communicating the two or more digital output signals.

Because of the above advantages provided by an apparatus according to the present invention, the present invention also relates to a method for measuring one or more physiological functions of a body comprising measuring of the one or more physiological functions using an apparatus according to the present invention.

Preferably, such method comprises multiple or continuous measurements of the one or more physiological functions providing a reliable representation or pattern of a physiological condition during a time course, such as one or more minutes, one or more hours, one or more days, one or more weeks, one or more months or even one or more years.

The term “multiple” as used herein is in generally understood to mean more than once in a certain time interval, or discontinuously. The term “continues” as used herein is in generally understood to mean a continuous measurement in a certain time interval.

The measurements generated using an apparatus according to the present invention provide an hitherto not achievable level of information regarding a physiological conditions, allowing precise, accurate, detailed and reliable diagnosis of a physiological conditions.

Hence, the present invention also relates to a method for analysis of one or more physiological functions of a body comprising multiple or continuous measuring of the one or more physiological functions using an apparatus according to the present invention, communicating the measurements to an external source, such as a microcomputer and analyzing the measurements using the external source, such as a microcomputer.

Hereafter, the above will be further exemplified by describing a detailed preferred embodiment of the present invention. It should be understood that this embodiment is not intended to limit the scope of the present invention but to provide the skilled person with a detailed embodiment allowing him with sufficient guidance to put other embodiments of the present invention in practice.

In this embodiment, the apparatus according to the present invention is in the form of a wrist watch, also designated as a PET Watch for measuring, on the wrist, of the heart rate and GSR (Galvanic Skin Response). Although watches like apparatuses for measuring the heart rate are already on the market, most of these systems use a chest belt or other external physiological detection means or sensors to measure heart rate. Because of this, these devices are not measurement devices per se, but are merely display and data storage devices.

The PET Watch according to the present invention measures the heart rate and GSR substantially directly on the wrist, i.e., the sensors incorporated in the watch are engaging the wrist area of the arm. This measurement according to the present invention is much more comfortable than other systems that use a chest belt or other extra sensors.

The heart rate is picked up at the wrist with the use of (multiple) pulse-oximetry sensors directly digitized without amplification.

The GSR is measured with two metal contact electrodes that are in contact with the skin.

A problem associated with reliably measuring pulse oximetry and GSR measurements at the wrist is that these measurements are rather sensitive to movement artefact. Therefore, preferably, an accelerometer is additionally used to detect motion.

When motion is below the critical predetermined threshold, indicating that a correct reliable pulse oximetry signal and GSR signal can be measured, a measurement is taken. Because a person is not moving continuously there are time intervals that there is no movement and good reliable heart rate and GSR readings can be done. For example, the PET Watch will be used to monitor emotional states (based on physiological data) of elderly people during the day. For this purpose, a couple of readings of heart rate and GSR per hour will be sufficient. This invention is for both real-time and offline applications.

The PET Watch has a Bluetooth wireless connection with a PC. Software on the PC can control the PET Watch by calling several functions through a DLL (Dynamic Link Library). When a connection between the software and PET Watch is established a command can be sent to the watch that starts a measurement. The measurement duration can be set by the PC. During a measurement, the accelerometer signal is measured and analyzed continuously. Heart rate and GSR data is measured when movement is below the critical threshold, indicating that a correct reliable pulse oximetry signal and GSR signal can be measured.

When the measurement time has elapsed, the measured data is analysed and processed by the DLL. Thereafter, a signal is given to the software that the measurement has finished. After the software received the “measurement finished” signal, other functions can be called that return data arrays with GSR, heart rate and accelerometer data. The software can further handle and process these signals and take actions desired for the application.

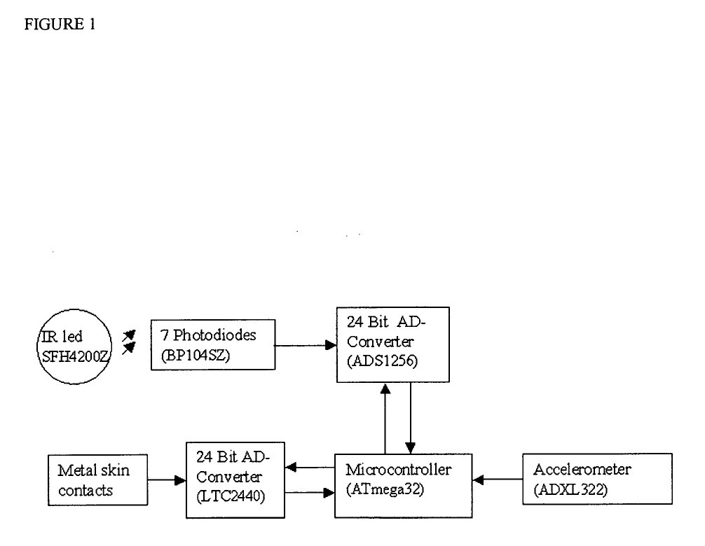

A schematic representation of this preferred embodiment of the present invention is depicted in FIG. 1.

In FIG. 1, The heart of the PET Watch is an Atmel Atmega32 (Atmel) microcontroller (1). The microcontroller (1) handles the communication with the microcomputer or personal computer (PC, not shown) through Bluetooth and controls all tasks and measurements.

For the pulse oximetry measurements, 7 photodiodes (BP104SZ, Osram), denoted with the numeral 2 are used. The cathodes of the photodiodes are connected to ground and the anodes of the photodiodes are immediately, without signal conditioning or amplification, except for a first order low pass pre-sampling filter (not shown), connected to the input of a high resolution AD-converter (ADS 1256 of Texas Instruments), denoted with the numeral 3. As a light source for pulse oximetry, an infrared LED (4) is used (SFH4200Z, Osram).

For the measurement of GSR, a measurement system using a high resolution AD-converter (LTC2440 of Linear Technology), denoted with the numeral 5 is used, that enables measurement of resistance from 0 Ohm to 5 Mohm between 2 metal contacts (6) on the skin. The physiological output signal of the metal skin contacts (6), without signal conditioning or amplification, except for a first order low pass pre-sampling filter (not shown), connected to the input of a high resolution AD-converter (5).

The accelerometer (7), used for motion detection, is the ADXL322JCP (two channels, dimensions (X,Y); Analog Devices.)

In order for the above apparatus to communicate with a microcomputer, the following DLL functions can be used:
int BQConnect(int port, int baudrate); Call this function at start up of the program to make a connection between the Watch and the software.
char * BQGetID(int handle); Get the ID of the hardware (is the Bluetooth address of the unit)
int BQInterval(int handle, int interval); Change the measurement time.
int BQShutDown(int handle); Start the measurement
int BQDataReady(int handle); Check and wait for data ready
call the following functions only after BQDataReady indicated that a measurement has finished:
int BQGetAccX(int handle, double * pValue, int length); Get the accelerometer data in the X-direction
int BQGetAccY(int handle, double * pValue, int length); Get the accelerometer data in the Y-direction
int BQGetECG(int handle, double * pValue, int length); Get the ECG data
int BQGetGSR(int handle, double * pValue, int length); Get the GSR data
int BQGetHRV(int handle, double * pValue, int length); Call this function after calling BQGetECG, this function returns the heart rate variability in the various measurement intervals.
After that, BQShutDown can be called again and the sequence will be repeated

From the 7 channels of the photodiodes (2), the most Suitable channels are selected according to a criterium Qj of lowest standard deviation and/or lowest mean second derivative on preliminary peak locations (t=tk) of the signals. These selected channels are then averaged and will be employed in final peak detection.

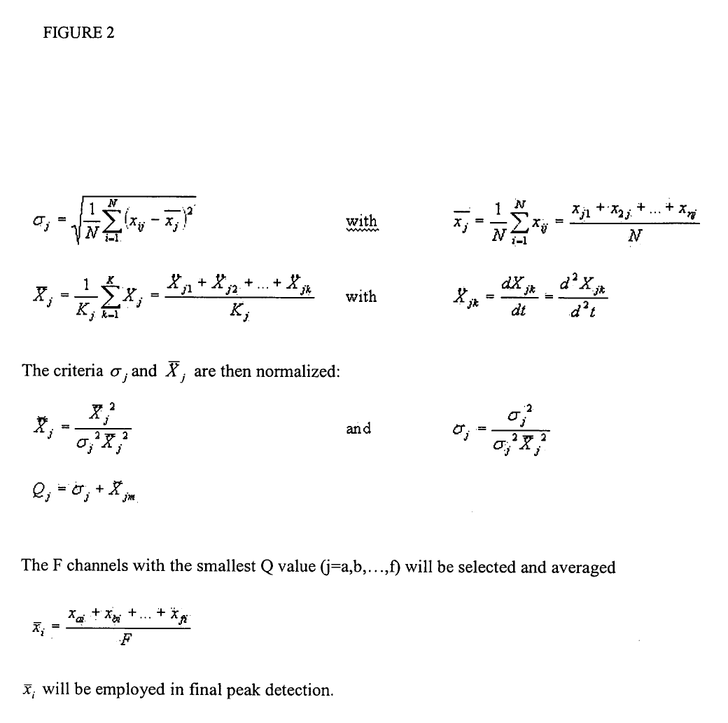

A possible operationalization of this would be that for every jth channel with N samples and k preliminarily detected peaks. The F channels with the smallest Q value (j=a,b, . . . ,f) will be selected and averaged. An example of such algorithm is summarized in FIG. 2

Although the present invention has been described with reference to a preferred embodiment thereof, it is apparent to the skilled person that a variety of modifications and changes may be made without departing from the scope of the present invention which is intended to be defined by the appended claims.

1. An apparatus for measurement of a physiological function of a body comprising; physiological function detection means responsive to a physiological signal and capable of generating a universal output signal corresponding to the physiological signal; an analog-digital converter capable of receiving the unamplified analog output signal and capable of generating a digital output signal corresponding to the unamplified analog output signal; control means capable of receiving and communicating the digital output signal.

2. An apparatus according to claim 1 further comprising movement detection means capable of generating a movement signal corresponding with movement of the body or a part thereof and the controls means are further capable of receiving the movement signal.

3. An apparatus according to claim 1, wherein the analog-digital converter is a high resolution analog-digital converter of 16 bit or higher.

4. An apparatus according to claim 1, wherein the analog-digital converter is a high resolution analog-digital converter of 24 bit or higher.

5. An apparatus according to claim 1, wherein the unamplified analog output signal is directly communicated to the analog-digital converter.

6. An apparatus according to claim 1, wherein the unamplified analog output signal is indirectly communicated through a low pass pre-sampling filter to the analog-digital converter.

7. An apparatus according to claim 1, wherein the physiological function detection means are suitable for detecting pulse oximetry and/or (phot)plethysmography and comprise an infrared or red light emitting source and a photodiode capable of detecting infrared or red light.

8. An apparatus according to claim 7, comprising two or more, preferably five or more, and most preferably seven photodiodes capable of detecting infrared or red light.

9. An apparatus according to claim 1, wherein the physiological function detection means are suitable for detecting the galvanic skin response and comprise two or more electrodes capable of measuring the resistance of the skin between the two or more electrodes.

10. An apparatus according to claim 1 comprising two or more physiological function detection means each responsive to a different physiological signal, two or more analog-digital converters corresponding with the two or more physiological signal detection means, and receiving means capable of receiving and communicating the two or more digital output signals.

11. Apparatus according to claim 10 comprising first physiological function detection means suitable for detecting pulse oximetry and/or (phot)plethysmography comprising an infrared or red light emitting source and one or more photodiodes capable of detecting infrared or red light; and second physiological function detection means suitable for detecting the galvanic skin response comprising two or more electrodes capable of measuring the resistance of the skin between the two or more electrodes.

12. Method for measuring one or more physiological functions of a body comprising measuring of the one or more physiological functions using an apparatus as claimed in claim 1.

13. Method according to claim 12 wherein the measuring comprises multiple or continuous measurements of the one or more physiological functions.

14. Method for analysis of one or more physiological functions of a body comprising multiple or continuous measuring of the one or more physiological functions using an apparatus as claimed in claim 1, communicating the measurements to an external source and analyzing the measurements using the external source.
